(19) United States
(12) Patent Application Publication
(10) Pub. No.: US 2025/0205417 A1
(43) Pub. Date: Jun. 26, 2025

(54) Wearable Drug Delivery Devices

(71) Applicant: Binary Biomedical, Inc., La Puente, CA (US)

(72) Inventor: Jason NG, La Puente, CA (US)

(73) Assignee: Binary Biomedical, Inc., La Puente, CA (US)

(21) Appl. No.: 18/982,052

(22) Filed: Dec. 16, 2024

Related U.S. Application Data
(60) Provisional application No. 63/613,229, filed on Dec. 21, 2023.

(51) Int. Cl. A61M 5/142; A61M 5/172

(52) U.S. Cl. CPC A61M 5/14248; A61M 5/1723; A61M 2205/3331

(57) ABSTRACT

A wearable drug delivery device comprises a removable pump assembly comprising a cartridge assembly and a controller module, a patch assembly, and a cannula assembly. The cartridge assembly may include a reservoir oriented such that the reservoir axis is parallel to the cannula axis. The reservoir may have a broad, flattened, low profile. The device may include an encoder and/or pressure sensor. The reservoir may be manufactured, for example, of deep-drawn sheet metal. The reservoir may have one or more internal ribs for maintaining piston alignment. The actuator assembly may include a large-diameter threaded actuator with a high thread density. The drive system may include a worm gear. Inserter systems, methods of use, and methods of manufacture are also disclosed.

### CROSS-REFERENCE TO RELATED APPLICATIONS

This application claims priority to U.S. Provisional Patent Application No. 63/613,229, filed Dec. 21, 2023, entitled “Wearable Drug Delivery Devices,” the entire contents of which are hereby incorporated herein by reference.

### TECHNICAL FIELD

The present disclosure is directed to wearable drug delivery devices and drug delivery systems including inserter systems for wearable drug delivery devices. The present disclosure is also directed to related methods of manufacture, assembly, and use of wearable drug delivery devices and drug delivery systems including inserter systems for wearable drug delivery devices.

### BACKGROUND

Wearable drug delivery devices are in common use. Such devices, in the form of wearable drug delivery pumps, are commonly used, for example, for automated, controlled delivery of insulin to patients with Type 1 or Type 2 diabetes.

Compared to multiple daily injections with an injector pen or syringe, wearable drug delivery pumps can provide greater flexibility of lifestyle. They can provide potentially better treatment control. For example, in the case of diabetes, they can provide potentially tighter blood glucose control without an increased risk of hypoglycemia, seizures, coma, or even death.

Wearable drug delivery pumps in current or prior use typically have one or more disadvantages. For example, some wearable drug delivery pumps have had a controller housing that attaches to a patient's body as well as one or more external tubes extending from the housing to a patch at an infusion site. Other wearable drug delivery pumps can be bulky, even without tubes. Prior designs can be inconvenient to deploy or wear and can be uncomfortable and aesthetically displeasing.

Wearable drug delivery pumps in current or prior use may have other disadvantages as well. For example, such prior devices may have one or more of the following disadvantages: a large size, a large footprint, a high profile, excessive weight, an internal reservoir volume that is too small, poor aesthetic appeal, uncomfortable, poor wearability, external tubes, inconvenience of use, difficulty of use, poor accuracy in drug delivery, poor precision in drug delivery, poor consistency in drug delivery, high cost, and/or difficult or expensive manufacturing.

### SUMMARY

In some embodiments, a wearable drug delivery device comprises a removable pump assembly comprising a cartridge assembly and a controller module, a patch assembly, and a cannula assembly comprising a cannula having a cannula axis. The cartridge assembly comprises a reservoir having a reservoir chamber and a reservoir axis. The reservoir may be oriented in the cartridge assembly such that when the pump assembly is attached to the patch assembly, the reservoir axis is aligned parallel to the cannula axis.

In some embodiments, the reservoir may have a broad, flattened, low profile. For example, the ratio of the diameter or width of the reservoir chamber to the depth of the reservoir chamber may be 2:1 or greater.

In some embodiments, the controller module housing may be translucent or transparent. The wearable drug delivery device may include an encoder and/or a pressure sensor.

In some embodiments, a wearable pump assembly comprises a cartridge assembly and a controller module. The cartridge assembly may have a reservoir manufactured from sheet metal. The cartridge assembly may have a reservoir manufactured using a deep draw process.

In some embodiments, the reservoir comprises one or more internal ribs. A piston disc may have one or more notches for accommodating the one or more ribs of the reservoir. A seal gasket around the perimeter of the piston disc may have one or more notches for accommodating the one or more ribs of the reservoir.

In some embodiments, a wearable pump assembly comprises a cartridge assembly and a controller module, wherein the cartridge assembly comprises a reservoir having a reservoir chamber, a piston disc, and an actuator assembly having a threaded actuator attached to the piston disc. The diameter of the threaded actuator attached to the piston disc may be one-quarter or more of the diameter or width of the reservoir chamber. The diameter of the threaded actuator attached to the piston disc may be one-half or more of the diameter or width of the reservoir chamber.

The cartridge assembly may further include an actuator retainer for maintaining alignment of the actuator assembly. The pump assembly may further include a drive system with a worm gear for driving the actuator assembly.

The threaded actuator may have a high thread density. For example, the threaded actuator may have a thread density of 80 threads per inch or higher.

In some embodiments, an inserter system for a drug delivery system comprises an inserter device and a cannula assembly loaded in the inserter device. The inserter device may comprise a retainer adapted to be selectively attached to a patch assembly. The inserter device may be adapted to launch the cannula assembly so that the cannula assembly connects to the patch assembly.

In some embodiments, a method of manufacturing a drug delivery system or a component for a drug delivery system is disclosed.

In some embodiments, a method of using a drug delivery system or a component for a drug delivery system is disclosed.

### BRIEF DESCRIPTION OF THE DRAWINGS

FIG. 1 shows a perspective view of components of an example embodiment of a removable pump assembly of a wearable drug delivery device.

FIG. 2A shows a top view of a cartridge assembly of the example embodiment of FIG. 1.

FIG. 2B shows a bottom view of the cartridge assembly of the example embodiment of FIG. 1.

FIG. 2C shows a perspective view of the cartridge assembly of the example embodiment of FIG. 1.

FIG. 2D shows another perspective view of the cartridge assembly of the example embodiment of FIG. 1.

FIG. 2E shows another perspective view of the cartridge assembly of the example embodiment of FIG. 1.

FIG. 3 shows an exploded view of the cartridge assembly of the example embodiment of FIG. 1.

FIG. 4A shows an exploded view of some of the components of the cartridge assembly of the example embodiment of FIG. 1, with certain components oriented to show certain features.

FIG. 4B shows another exploded view of some of the components of the cartridge assembly of the example embodiment of FIG. 1.

FIG. 4C shows a perspective view of some of the components of the cartridge assembly of the example embodiment of FIG. 1.

FIG. 4D also shows a perspective view of some of the components of the cartridge assembly of the example embodiment of FIG. 1.

FIG. 5A shows a top view of a reservoir of the cartridge assembly of the example embodiment of FIG. 1.

FIG. 5B shows a side view of the reservoir of the cartridge assembly of the example embodiment of FIG. 1.

FIG. 5C shows a perspective view, generally from the bottom, of the reservoir of the cartridge assembly of the example embodiment of FIG. 1.

FIG. 5D shows a side view of a reservoir with a piston disc.

FIG. 6A shows a perspective view of a controller module of the example embodiment of FIG. 1, with an upper housing separated from the remaining components.

FIG. 6B shows a perspective view, generally from the bottom, of the controller module of the example embodiment of FIG. 1.

FIG. 6C shows the inside of the upper housing of the controller module of the example embodiment of FIG. 1.

FIG. 7A shows an exploded view of components of the example embodiment of FIG. 1, showing the positioning of the cartridge assembly within the controller module.

FIG. 7B shows a perspective view of components of the example embodiment of FIG. 1, showing the positioning of the cartridge assembly within the controller module, with certain components of the controller module separated from the remaining components.

FIG. 7C shows a perspective view of components of the example embodiment of FIG. 1, with the lower housing of the controller module removed.

FIG. 8A shows a perspective view, generally from the bottom, of a lower housing of the controller module of the example embodiment of FIG. 1.

FIG. 8B shows an exploded perspective view, generally from the top, of an example embodiment of a patch assembly of a wearable drug delivery device, the patch assembly being usable with the removable pump assembly of FIG. 1.

FIG. 8C shows a top view of the patch assembly of FIG. 8B.

FIG. 8D shows a side view of a locking stud of the patch assembly of FIGS. 8B-8C.

FIG. 9A shows an example embodiment of certain components of an inserter system.

FIG. 9B shows another view of the example embodiment of FIG. 9A.

FIG. 9C shows a bottom view of a retainer component of the embodiment of FIG. 9A.

FIG. 10A shows an exploded view of certain components of the example embodiment of FIG. 9A.

FIG. 10B shows an example embodiment of a cannula assembly of a wearable drug delivery device, the cannula assembly being usable with the patch assembly of FIGS. 8B-8C and the removable pump assembly of FIG. 1.

FIG. 11A shows a cross-sectional view of certain components of the example inserter system of FIG. 9A, prior to attaching a cannula assembly to a patch assembly.

FIG. 11B shows a cross-sectional view of the cannula assembly from FIG. 11A attached to a patch assembly.

FIG. 12A shows a partially-exploded view of an example inserter system, with an inserter device in a first position.

FIG. 12B shows a perspective view of the example inserter system of FIG. 12A, with the inserter device in a second position.

FIG. 12C shows an example drug delivery system, after deployment of the cannula assembly and separation of the inserter device from the patch-cannula assembly.

FIG. 12D shows the underside of the patch-cannula assembly after deployment of the cannula assembly.

FIG. 12E shows the assembled wearable drug delivery device, with the upper housing and certain components of the controller module removed.

### DETAILED DESCRIPTION

The following is a detailed description of the illustrated embodiment(s) and certain variations of such embodiment(s). Numerous other variations are possible within the scope of the disclosure, as would be understood by persons of ordinary skill in the art.

Various embodiments of drug delivery systems are described herein. In one example embodiment, the drug delivery system may be supplied to a user (patient, health care practitioner, etc.) as: (i) an inserter system, which includes an inserter device, a cannula assembly loaded in the inserter device, and a patch assembly attached to (or attachable to) the inserter device, and (ii) a removable pump assembly (assembled, or as components adapted to be assembled). The inserter device may be used to facilitate placement of the patch assembly on the patient and to launch the cannula assembly so that a cannula of the cannula assembly is inserted into the patient through the patient's skin and so that the cannula assembly is connected with the patch assembly. The components of the removable pump assembly may include a cartridge assembly and a controller module. The cartridge assembly and controller module may be supplied to the user as separate components or assembled together as a removable pump assembly. Once the inserter device has been used to place the patch assembly on the patient and to launch the cannula assembly so that the cannula is inserted into the patient, the inserter device may be removed and, optionally, disposed of. The remaining components—the patch assembly with the connected cannula assembly and the removable pump assembly (cartridge assembly and controller module)—together form a wearable drug delivery device for delivery of a selected drug in a controlled manner to the patient.

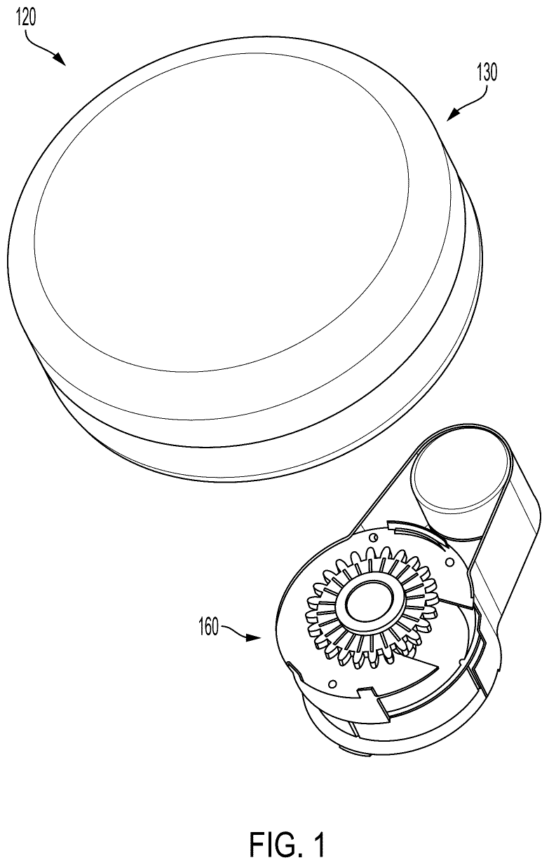

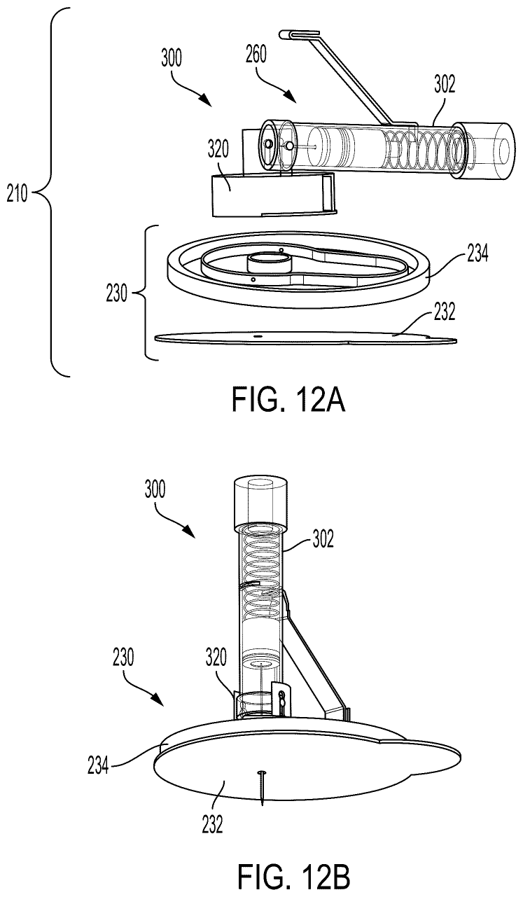

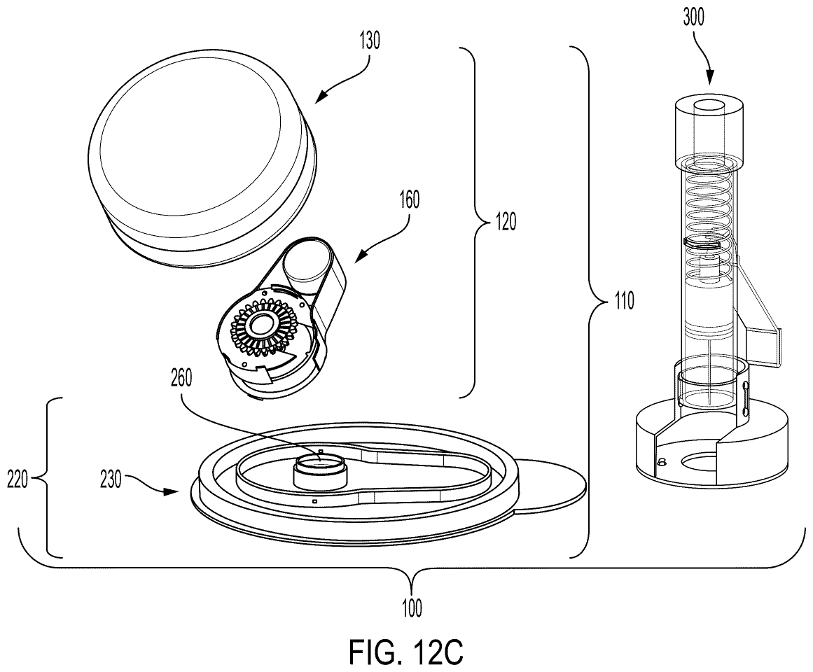

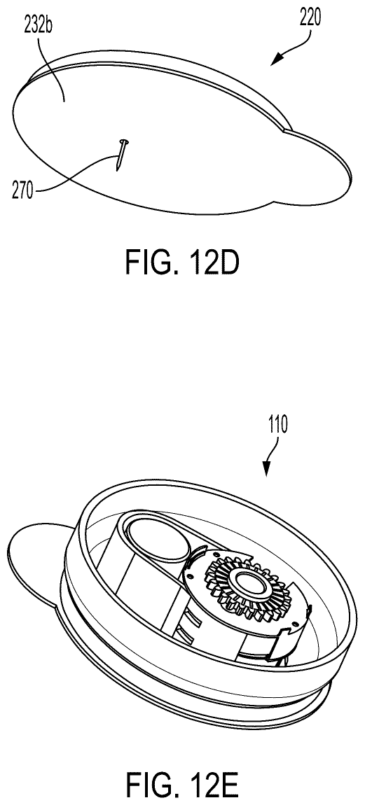

FIG. 1 shows components of an example embodiment of a removable pump assembly 120 of an example wearable drug delivery device 110 (see FIG. 12C). The illustrated removable pump assembly 120 includes a controller module 130 and a cartridge assembly 160.

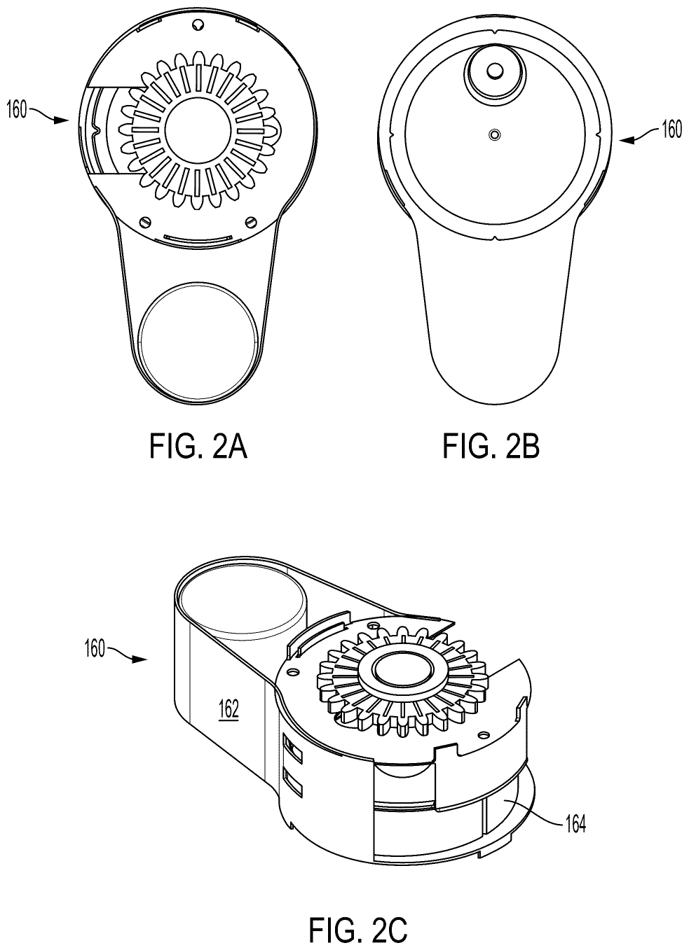

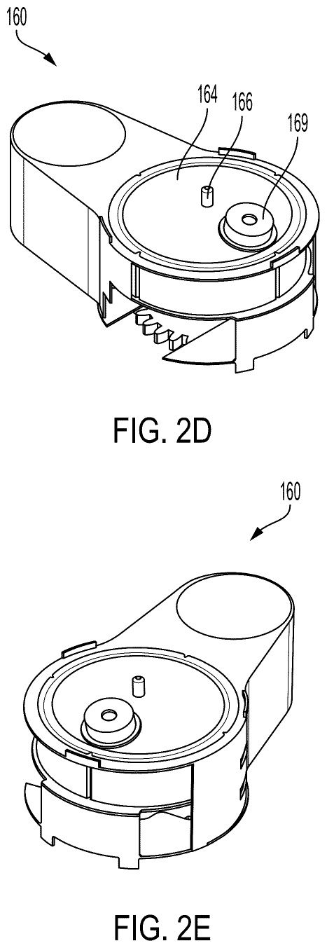

FIG. 2A shows a top view of the cartridge assembly 160 of the example embodiment of FIG. 1. FIG. 2B shows a bottom view of the cartridge assembly 160. FIGS. 2C-2E show perspective views of the cartridge assembly 160.

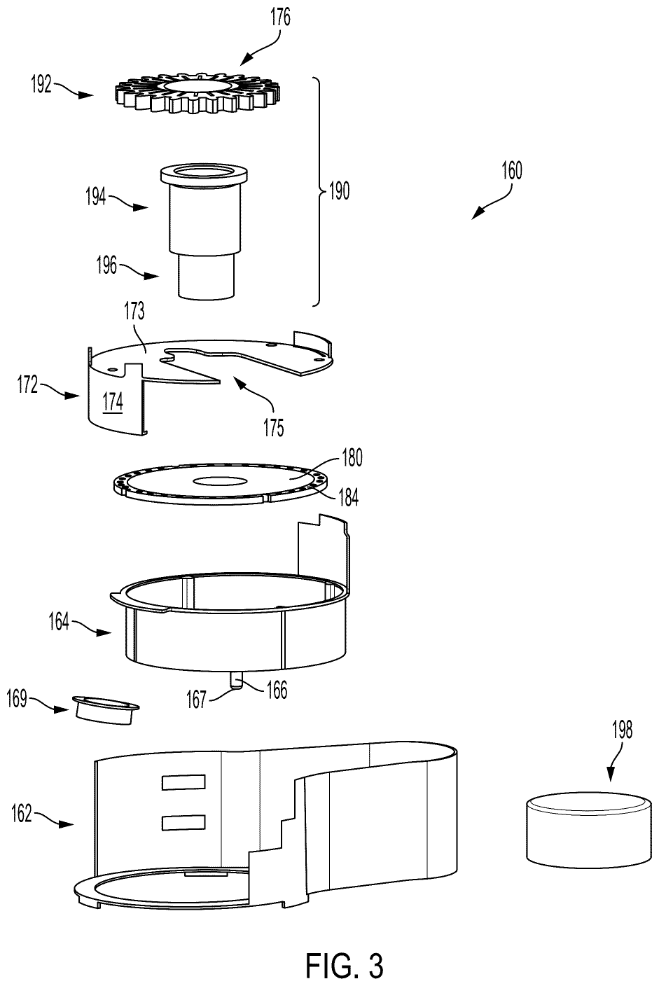

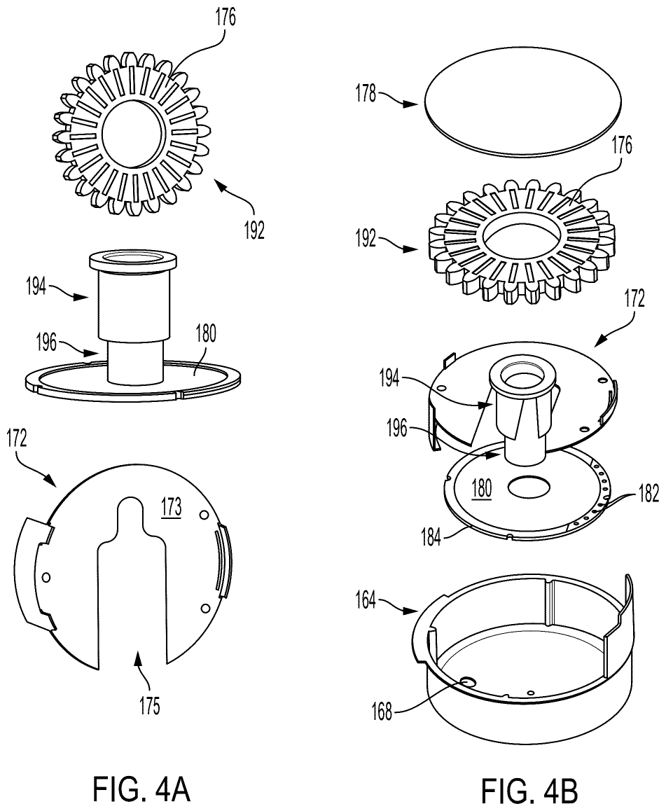

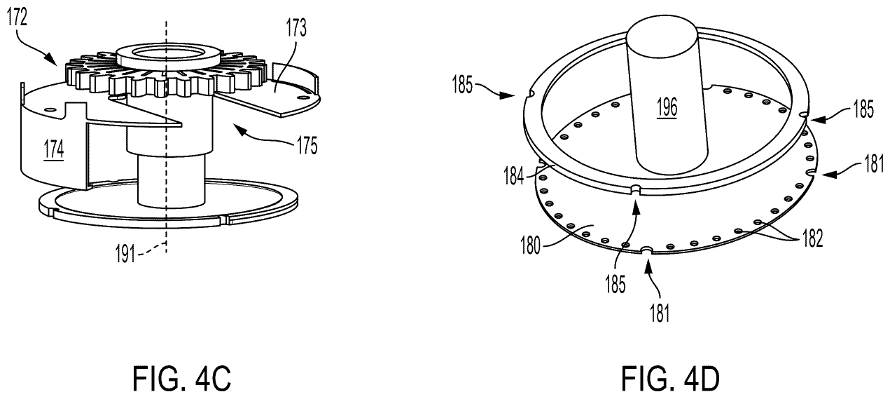

FIG. 3 shows an exploded view of the cartridge assembly 160. FIGS. 4A and 4B show exploded views of some of the components of the cartridge assembly 160, with certain components oriented to show certain features. FIGS. 4C and 4D show perspective views of some of the components of the cartridge assembly 160.

As illustrated in the drawings, the example cartridge assembly 160 has a housing 162 in which components of the cartridge assembly 160 are located. The cartridge assembly 160 includes a reservoir 164 located in the housing.

The reservoir 164 has an internal reservoir chamber that holds the drug fluid to be delivered to the patient. In this embodiment the reservoir 164 is shaped generally in the form of a cylinder, but many other shapes are possible. The bottom of the reservoir 164 has a microminiature tube 166 with a fluid outlet port 167 at its distal end. The bottom of the reservoir also has a fill port 168 which is an opening in the bottom wall of the reservoir. The fill port 168 is covered by a fill septum 169, which may be an elastomer such as silicone or rubber.

The top of the reservoir 164 has an opening that accommodates a movable piston 180, which in the illustrated embodiment is in the form of a disc. In the illustrated embodiment of FIG. 4D, the piston disc 180 is a thin, flat disc. The piston disc 180 may be in one plane or may be slightly domed or convex to conform to the shape of the bottom wall 164b of the reservoir 164 (described below). The piston disc 180 has a gasket 184 encircling its outer periphery, around the circumference. When assembled, the gasket 184 forms a seal with the inner wall of the reservoir 164, such that the reservoir 164 and piston disc 180 and gasket 184 form an enclosed drug fluid chamber open only at the outlet port 167 (the fill port 168 is kept closed by the septum 169, but the fill port 168 allows access to the reservoir 164 for filling, as described below).

To facilitate the connection between the piston disc 180 and the seal gasket 184, the disc 180 may have one or more holes 182 through it. In the illustrated example, the disc 180 has a plurality of holes 182 aligned in a circle or along one or more arcs near the outer edge of the disc 180. FIG. 4D shows the holes 182; FIG. 4B has part of the gasket 184 removed to show the position of some of the holes 182. The gasket 184 may be formed by over-molding it over the outer edge of the disc 180, such that the material of the gasket 184 passes through the holes 182. In this way, the solidified gasket 184 is strongly bonded to the disc 180. This helps prevent the gasket 184 from being dislodged from the disc 180 when it slides against the inner wall of the reservoir 164. This also facilitates a proper and uniform seal and helps prevent leaks.

The disc 180 may have one or more notches 181 in its outer edge. The seal gasket 184 may correspondingly have one or more notches 185, corresponding to the notches 181 of the disc 180. The notches 181, 185 accommodate ribs 170 of the inside of the reservoir 164, as described below.

The piston disc 180 is advanced in the reservoir 164 to dispense the drug fluid to the patient by action of an actuator assembly 190. The actuator assembly 190 includes a gear 192 which in the illustrated embodiment is in the form of a disc gear, also referred to as a spur gear or toothed wheel, with gear teeth around the outer periphery of the disc-shaped gear. The gear 192 is connected to a first threaded actuator 194, which in the illustrated embodiment is a female-threaded tube, i.e., a tube with a helical thread along the inside surface of the tube. A second threaded actuator 196 is connected to the piston disc 180. In the illustrated embodiment, the second threaded actuator 196 is a male-threaded rod, i.e., a rod with a helical thread along its outside surface. When assembled, the rod fits within the tube with the external threads of the rod threadingly-engaged with the internal threads of the tube. In an alternative embodiment, the female-threaded actuator and the male-threaded actuator are reversed, such that the first threaded actuator connected to the gear 192 is a male-threaded rod, and the second threaded actuator connected to the disc 180 is a female-threaded tube.

As described in more detail below, each of the threaded elements of the actuator assembly 190, i.e., the first threaded actuator 194 and the second threaded actuator 196, has a very precise, fine, helical thread. The result is a precision threaded actuator that makes very precise, small movements of the piston disc 180 for precision drug delivery.

In the illustrated embodiment, the example cartridge assembly 160 further has an actuator retainer 172 which serves to keep components of the actuator assembly 190 positioned and aligned. In this example, the actuator assembly components are aligned vertically with respect to the piston disc 180. The first threaded actuator 194 and the second threaded actuator 196 are aligned along a central longitudinal axis 191 running through the rod and tube. The axis 191 is the axis of rotation for the gear 192 and the first threaded actuator 194. The axis 191 is perpendicular to the general plane of the piston disc 180. The actuator retainer 172 helps keep components of the actuator assembly 190 aligned so that the axis 191 is aligned with (parallel to and/or colinear with) an axis 165 of the reservoir 164.

In the illustrated embodiment, the actuator retainer 172 is a bracket that has a central plate 173, an extension 174, and an opening 175 in the plate 173. When the cartridge assembly 160 is assembled, the extension 174 is supported relative to the housing 162 so that the actuator retainer 172 is held stable. The actuator assembly 190 is positioned within the opening 175 in the plate 173 so that it is braced by the edge(s) of the plate 173 around the opening 175. In this way, the actuator retainer 172 helps keep components of the actuator assembly 190 aligned so that the axis 191 is aligned with an axis 165 of the reservoir 164. Thus, the actuator retainer 172 supports the axial (vertical) directional alignment and movement of the threaded actuator(s) 194, 196 with accuracy and helps prevent the piston disc 180 from tilting relative to the reservoir 164 during the fluid dispensing process.

The actuator retainer 172 also can help hold the actuator assembly 190 together. In some embodiments, the plate 173 of the actuator retainer 172 can serve as a support below the toothed wheel 192. Thus, the actuator retainer 172 can help resist downward external pressure on the actuator assembly 190 and unwanted dispensing of drug fluid from the reservoir 164. For example, the actuator retainer 172 can resist external pressures, such as during airplane flights, which can cause unwanted movements of the actuator assembly 190.

In the illustrated embodiment, the example cartridge assembly 160 further includes an encoder 176 that moves with the toothed wheel 192. The encoder 176 may be a disc connected to the toothed wheel 192. The encoder 176 has indicia in the form of markings, electromagnetic elements, optical elements, etc., that can be detected by a reader 156 to provide feedback on the movement and/or position of the actuator and, correspondingly, the piston disc 180. While the encoder 176 may be a disc connected to the toothed wheel 192, in alternative embodiments the encoder 176 may be indicia located directly on the gear 192 or on the first threaded actuator 194 or another rotating part of the actuator assembly 190. As described below, the encoder 176 cooperates with an encoder reader 156 to provide real-time feedback of the position of the toothed wheel 192 and, accordingly, the piston disc 180, to provide confirmation of the position of the piston disc 180 (and/or to indicate any error in, and/or to allow for correction of, the position of the piston disc 180).

In the illustrated embodiment, the example cartridge assembly 160 may further include a pressure sensor 178. The pressure sensor 178 may be a component of the cartridge assembly 160 and/or the controller module 130. The pressure sensor 178 is positioned to sense an upward force on the actuator assembly 190. For example, if there is a clog in the outlet port 167 or some other occlusion or issue with fluid exiting the reservoir 164, or if the piston disc 180 reaches the bottom of the reservoir 164, further actuation of the actuator assembly 190 accompanied by a restriction on movement of the piston disc 180 will cause an upward force on the actuator assembly 190. The pressure sensor 178 can detect this force to detect the occlusion (or other issue). In some embodiments, the pressure sensor 178 contacts a side of the disc gear 192. In other embodiments, one or more components may be located between the pressure sensor 178 and the disc gear 192 or other component of the actuator assembly 190.

The example cartridge assembly 160 may further include a battery 198. The battery 198 may be a component of the cartridge assembly 160 and/or the controller module 130. The battery 198 provides electrical power as needed, for example for driving the actuator assembly and/or for one or more sensors/readers (e.g., encoder, pressure sensor, etc., and/or associated readers).

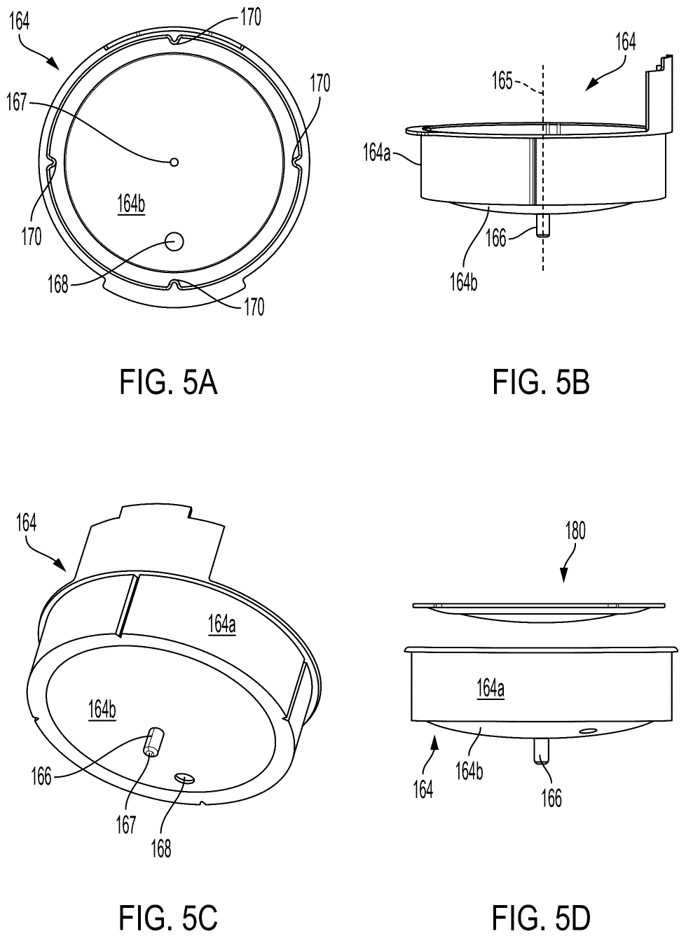

FIGS. 5A, 5B, and 5C show a top view, side view, and perspective view of the example reservoir 164. The reservoir 164 has a side wall 164a and a bottom wall 164b. The side wall 164a may define any suitable cross-sectional shape for the reservoir chamber, such as generally circular (as illustrated), elliptical, rectangular, square, hexagonal, octagonal, etc. The bottom wall 164b may have any suitable shape, such as flat, conical, dome-shaped, convex, etc. In some embodiments, the piston disc 180 may be shaped to correspond to the shape of the bottom wall 164b, such that when the actuator is fully deployed, the piston disc 180 is flush against the bottom wall 164b to expel all or substantially all of the fluid medicament from the reservoir 164. FIG. 5D shows a side view of a reservoir with a dome-shaped bottom wall 164b along with a piston disc 180 similarly dome-shaped to correspond to the shape of the bottom wall 164b.

In the illustrated example embodiment, the inner side of the side wall 164a of the reservoir 164 has inwardly-extending ribs 170. The illustrated embodiment shows four ribs equally-spaced, at 90 degree increments, around the inner periphery of the side wall 164a; however, any suitable number and placement of ribs may be used. For example, the reservoir may have one rib, two ribs 180 degrees apart, three ribs 120 degrees apart, five ribs 72 degrees apart, etc. Unequal spacings may also be used. In one alternative example, eight ribs are arranged in pairs, with the pairs spaced at 90 degree increments. When the cartridge assembly 160 is assembled, the ribs 170 are accommodated in the notches 181, 185 of the piston disc 180 and gasket 184, respectively. In this way, the ribs 170 serve as guides for the piston disc 180, to help keep the piston disc 180 from tilting during the dispensing process.

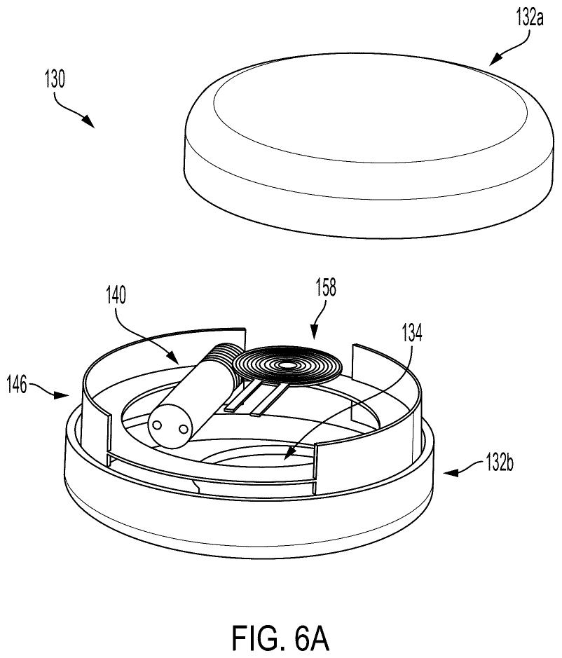

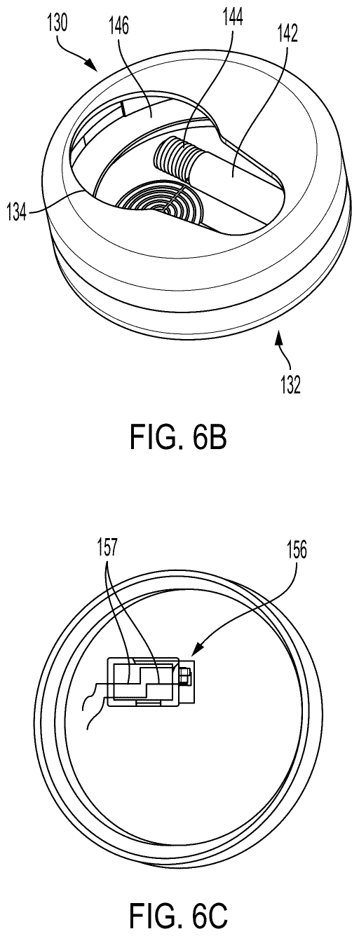

FIGS. 6A and 6B show perspective views of a controller module 130 of the example removable pump assembly 120. FIG. 6A shows the controller module with the upper housing 132a separated from the remaining components. FIG. 6C shows the inside of the upper housing 132a.

As illustrated in the drawings, the example controller module 130 has a housing 132 in which components of the controller module 130 are located. The housing 132 includes a top housing 132a and a lower housing 132b that fit together to form the housing 132. The lower housing 132b has an opening 134 for the controller module 130 to be placed over the cartridge assembly 160 to connect the controller module 130 and the cartridge assembly 160 together.

The example controller module 130 includes, inside the housing 132, an electromechanical drive system 140. The electromechanical drive system 140 is used to drive the actuator assembly 190 of the cartridge assembly 160. The electromechanical drive system 140 includes a motor 142 that drives a gear 144. In this example, the gear 144 is a worm gear 144 that engages the teeth of the toothed wheel 192 when the cartridge assembly 160 is assembled within the controller module 130. In alternative embodiments, other arrangements may be used for driving the actuator assembly 190.

The example controller module 130 also includes a printed circuit board assembly (PCBA) 146. The PCBA 146 may include electronics (hardware including one or more microcontrollers and/or other microchips, running firmware and/or software) for various functions, such as receiving input data from an external source, such as from a device (e.g., smart phone, tablet, computer, etc.) or other source sending signals for controlling the functioning of the wearable drug delivery device (as described below); receiving input data from a source internal to the wearable drug delivery device, such as the pressure sensor(s), the encoder(s), and/or one or more other sensors; receiving input data from the battery, e.g., detecting the battery level; processing input data; activating/controlling the electromechanical drive system for dispensing drug; and/or activating one or more signaling devices such as an alarm, light, and/or vibration mechanism. Connections may be provided such that when the cartridge assembly 160 is assembled within the controller module 130 the PCBA 146 is connected to the battery 198 to receive power from the battery 198. One or more wires or connections (not numbered) may electrically connect the drive motor 142 to the PCBA 146. The PCBA 146 is illustrated as extending around the inside periphery of the housing 132, encircling other components. The PCBA 146 may extend fully or only part way around the inside of the housing 132. Other arrangements and placements for the PCBA 146 are possible in alternative variations.

In the illustrated embodiment, the example controller module 130 may further include a pressure sensor 158, which may be used in addition to or in place of pressure sensor 178. Like the pressure sensor 178, the pressure sensor 158 may be positioned to sense an upward force on the actuator assembly 190. The pressure sensor 158 may be positioned similarly to the pressure sensor 178, as described above. One or more wires or connections 159 may connect the pressure sensor 158, 178 to the PCBA 146.

The microcontroller or other electronics on the PCBA 146 may be programmed to correlate detected electrical signals (voltages) from the pressure sensor(s) 158, 178 to corresponding associated pressures and to send one or more signals when the detected pressure is outside of a certain range. For example, a pressure exceeding a programmed maximum pressure can indicate that an occlusion is blocking or restricting the outflow of the drug fluid, or that the piston disc has reached the bottom of the reservoir, or that there is some malfunction occurring. When a pressure exceeding the programmed maximum pressure is detected, the microcontroller or other electronics can activate one or more signals, e.g., sounding a buzzer or other alarm in the device, lighting or flashing one or more lights (LEDs) on the device, and/or activating a vibration mechanism in the device that causes the device to vibrate. For example, a maximum pressure threshold may be programmed as 10 grams, 12 grams, 15 grams, or another amount of force, dependent upon the embodiment and application.

The example controller module 130 may further include an encoder reader or detector 156. In one example, the encoder reader 156 is an optical encoder reader. The encoder reader 156 is positioned on the inside of the upper housing 132a. The encoder reader 156 is positioned such that when the cartridge assembly 160 is assembled within the controller module 130 the encoder reader 156 is capable of reading the encoder 176. One or more wires or connections 157 may electrically connect the encoder reader 156 to the PCBA 146.

The encoder reader 156 detects movement of the encoder 176, sending signals to the electronics on the PCBA 146. These signals indicate how far the encoder 176 has turned, which indicates how far the rotating components of the actuator assembly 190 have turned and thus how far the piston disc 180 has advanced into the reservoir 164. In this way, the electronics receive information regarding how much drug fluid has been dispensed from the reservoir.

During operation of the wearable drug delivery device, the dispensing of the drug fluid may be either open-loop or closed-loop. In an open-loop embodiment, the electronics receive information on the amount of drug fluid to dispense in a particular dose and then send signals to the motor 142 for the motor to move in an amount corresponding to the amount of drug fluid to be dispensed. In a closed-loop embodiment, the electronics receive information on the amount of drug fluid to dispense in a particular dose and then send signals to the motor 142 for the motor to move to dispense drug fluid, while feedback signals from the encoder reader 156 provide information on how far the actuator assembly 190 has moved. When these feedback signals indicate that the intended amount of drug fluid has been dispensed, the electronics stop driving the motor 142. In some embodiments, the encoder reader 156 and the encoder 176 may also be used to determine the total amount of drug fluid dispensed from the reservoir 164, and the electronics may use such information to activate one or more signals when the drug fluid in the reservoir 164 has been depleted (or is about to be depleted), as described above (e.g., sounding a buzzer or other alarm in the device, lighting or flashing one or more lights (LEDs) on the device, and/or activating a vibration mechanism in the device that causes the device to vibrate).

As mentioned above, the electronics may also receive input data from the battery, e.g., detecting the battery level. The electronics may send a signal (e.g., sound, light, vibration) to indicate a low battery level. The electronics may also send a signal when they detect a malfunction, such as the motor not moving, or the actuator movement as detected from the encoder not matching up with the amount the motor was intended to move based on driving signals sent to the motor, or some other issue.

The wearable drug delivery device 110 may include an antenna for receiving input signals from an external source, such as a smart phone, tablet, computer, or remote source. The signals may be received in any suitable manner, such as Bluetooth, RF technology, WiFi, cellular signals, or other wireless signaling. The antenna may be located on the PCBA 146, or it may be a separate component connected to the PCBA 146. In one example, traces on the PCBA 146 constitute the antenna.

In some embodiments, the wearable drug delivery device 110 may be used without connection to any glucose monitoring device. In other embodiments, the wearable drug delivery device 110 may include a glucose sensor. In other embodiments, the wearable drug delivery device 110 may be configured to receive signals from an external glucose sensor, which may be worn elsewhere on the patient's body, or which may otherwise be used to detect the patient's glucose level (e.g., from reading a sample). The electronics on the PCBA 146 may receive input signals from the glucose sensor and may use such signals in controlling the delivery of drug fluid to the patient (e.g., determining dosage amounts, determining dosage timing, dispensing additional doses, withholding doses, etc.).

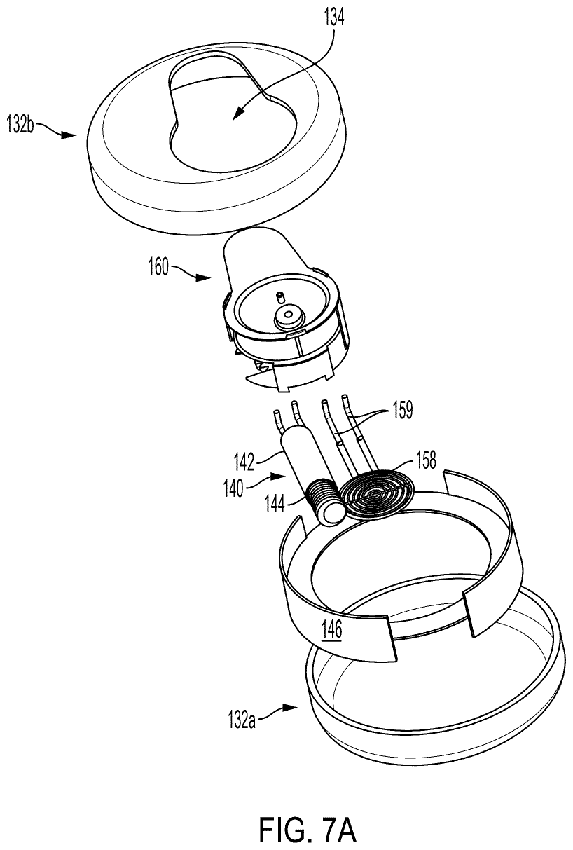

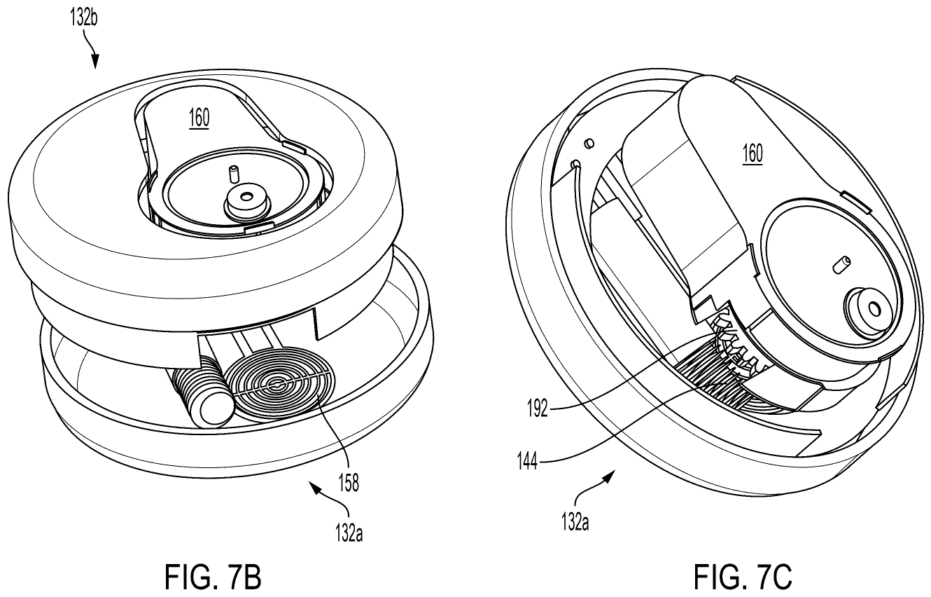

FIG. 7A shows an exploded view of components of the example removable pump assembly 120, showing the positioning of the cartridge assembly 160 within the controller module 130. FIG. 7B shows a perspective view of the removable pump assembly 120 including the cartridge assembly 160 within the controller module 130, with certain components of the controller module 130 separated from the remaining components. FIG. 7C shows a perspective view with the lower housing 132b of the controller module 130 removed.

In the illustrated embodiment, the opening 134 in the housing 132 of the controller module 130 is shaped and sized to correspond to the shape and size of the cartridge assembly 160. The opening 134 is slightly larger than the cartridge assembly 160 to facilitate placement of the cartridge assembly 160 within the controller module 130. In the illustrated example, the opening 134 and the cartridge assembly 160 generally have the shape of a keyhole, with a circle (where the reservoir 164 is located) and a projection of narrower width extending from the circle. Many other shapes are possible in alternative variations.

The cartridge assembly 160 and/or the controller module 130 may include locking elements (not shown) that keep the cartridge assembly 160 and the controller module 130 held together as a single removable pump assembly 120, while allowing the cartridge assembly 160 to be easily removed from the controller module 130. Such locking elements may include, but are not limited to, resilient tabs, slots, openings, snaps, etc.

When the cartridge assembly 160 is assembled within the controller module 130, the actuator assembly 190 is aligned with the drive system 140 so that the drive system 140 can drive the actuator assembly 190. In the illustrated example, when the cartridge assembly 160 is assembled within the controller module 130, the gear 192 of the actuator assembly engages the gear 144 of the drive system 140. That is, the spiral thread of the worm gear 144 is engaged with the teeth of the disc gear 192, so that driving the worm gear 144 causes the disc gear 192 to turn.

Similarly, when the cartridge assembly 160 is assembled within the controller module 130, other components may align, such as sensors or readers. For example, when the cartridge assembly 160 is assembled within the controller module 130, the encoder reader 156 is positioned adjacent or above the encoder 176 such that the encoder reader 156 is capable of reading the encoder 176. Also, when the cartridge assembly 160 is assembled within the controller module 130, the pressure sensor 158 and/or 178 is positioned to sense an upward force on the actuator assembly 190, as described above.

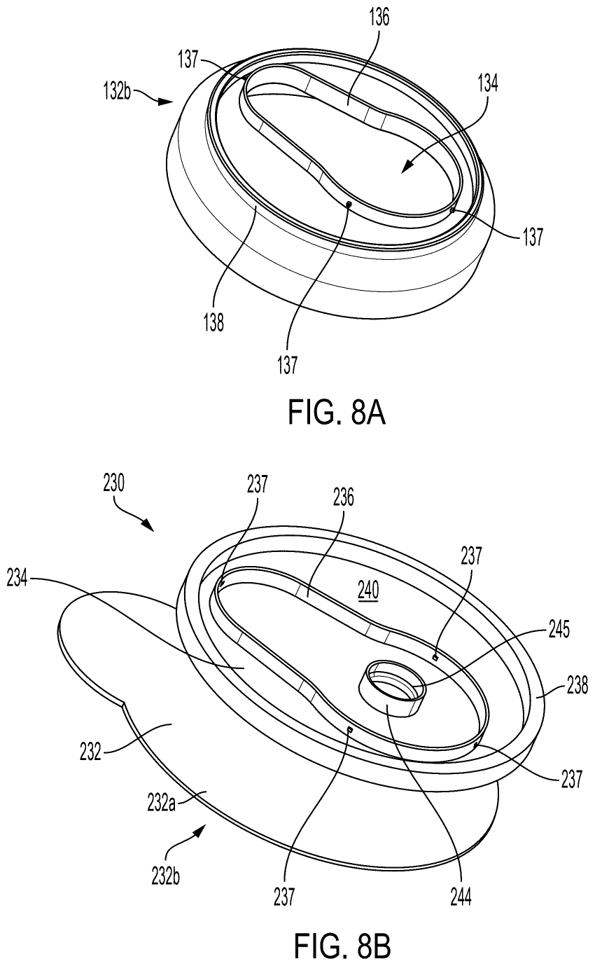

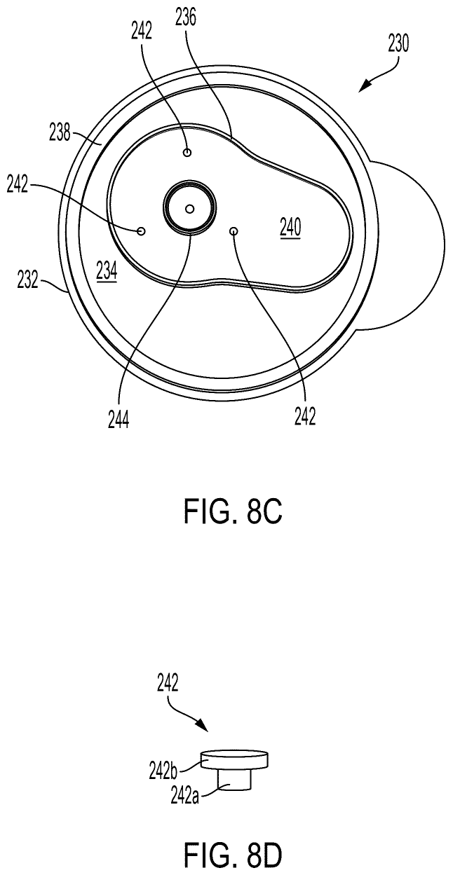

FIG. 8A shows a view of the bottom of the lower housing 132b of the controller module 130. Certain details visible in FIG. 8A are omitted from certain other figures for clarity. As can be seen in FIG. 8A, the lower housing 132b has a raised ridge 136 in the form of a wall or lip located near or around the opening 134. The ridge 136 includes locking elements 137 that cooperate with locking elements 237 (described below) of a ridge 236 of a patch assembly 230 that keep the removable pump assembly 120 connected to the patch assembly 230, while allowing the removable pump assembly 120 to be easily removed from the patch assembly 230. Such locking elements may include, but are not limited to, resilient tabs, slots, openings, snaps, etc. In the illustrated embodiments, the locking elements 137 are tabs (which may be, for example, resilient and/or configured for a press or interference fit), while the locking elements 237 are openings configured to receive the tabs 137. When the removable pump assembly 120 is pressed with sufficient force into place on the patch assembly 230, the tabs 137 snap into the openings 237 and keep the removable pump assembly 120 secured to the patch assembly 230. The removable pump assembly 120 can be removed from the patch assembly 230 by applying a sufficient upward force on the removable pump assembly 120 while holding the patch assembly 230, thereby pulling the tabs 137 out of the openings 237 and releasing the removable pump assembly 120 from the patch assembly 230. While the illustrated embodiment includes four sets of locking elements 137, 237 positioned as shown, any suitable number and positioning of locking elements 137, 237 may be used. Other mechanisms of releasable attachment between the removable pump assembly 120 and the patch assembly 230 may be used (e.g., twisting, etc.).

As shown in FIG. 8A, the lower housing 132b also has a raised ridge 138 in the form of a wall or lip located near or around the lower periphery of the lower housing 132b. The ridge 138 cooperates with a ridge 238 on the patch assembly 230 in order to facilitate proper placement of the pump assembly 120 on the patch assembly 230. The ridge 136, the ridge 138, the ridge 236, and/or the ridge 238 may facilitate waterproofing of the device, to help prevent water or other liquids from damaging internal components of the pump assembly 120. The use of a circular profile with a circular ridge such as ridge 138 and/or ridge 238 can facilitate waterproofing because of the even distribution of pressure around the ridge. One or more sealing elements, such as an elastomeric seal or gasket, may be provided along one or more of the ridges 136, 236, 138, 238 (e.g., along the inside, outside, and/or top of the ridge) to facilitate sealing.

FIG. 8B shows an exploded view of an example embodiment of a patch assembly 230 of a wearable drug delivery device. FIG. 8C shows a top view of the patch assembly 230. FIG. 8D shows a side view of a locking stud 242 of the patch assembly 230 (the locking studs are not shown in FIG. 8B).

The patch assembly 230 is usable with the removable pump assembly 120 of FIG. 1. In the illustrated example, the patch assembly 230 includes a patch 232 and a patch mount 234. The patch 232 is a sheet of material adapted to be adhered to a patient's body. While the patch 232 is relatively flexible, the patch mount 234 is relatively inflexible, made for example from injection-molded or 3D-printed plastic. The patch mount 234 may be adhered or otherwise secured to an upper side 232a of the patch 232. The upper side 232a may include an adhesive material or coating for securing the patch 232 to the patch mount 234. The lower side 232b of the patch 232 may also include an adhesive material or coating for securing the patch 232 to the patient's skin. The adhesive may be over all, substantially all, or a portion or portions of the lower side 232b. The adhesive on the lower side 232b may be covered by a removable paper to be removed just prior to adhering the patch assembly 230 to the patient.

The patch mount 234 has a bottom plate 240 that is secured to the patch 232. The bottom plate 240 has a raised inner ridge 236 in the form of a wall or lip that is shaped and sized so that the ridge 136 of the controller module 130 can be received within the ridge 236. In certain embodiments, such as the illustrated embodiment, the ridge 236 receives the ridge 136 securely, with little or no gap between the ridges 136, 236, so that the ridges help prevent the pump assembly 120 from movement relative to the patch assembly 230. The ridge 236 includes locking elements 237 that cooperate with locking elements 137 of the controller module 130 to keep the removable pump assembly 120 connected to the patch assembly 230, while allowing the removable pump assembly 120 to be easily removed from the patch assembly 230. As described above, such locking elements may include, but are not limited to, resilient tabs, slots, openings, snaps, etc. In the illustrated embodiment, the locking elements 237 are openings configured to receive the tabs 137 of the controller module 130.

The bottom plate 240 of the patch mount 234 also has a raised outer ridge 238 in the form of a wall or lip that is shaped and sized so that the ridge 138 of the controller module 130 can be received within the ridge 238. In certain embodiments, such as the illustrated embodiment, the ridge 238 receives the ridge 138 securely, with little or no gap between the ridges 138, 238, so that the ridges help prevent the pump assembly 120 from movement relative to the patch assembly 230. Although not shown, the ridge 138 and/or the ridge 238 may include one or more locking elements to facilitate the removable connection of the pump assembly 120 to the patch assembly 230.

The patch assembly 230 may further include locking elements 242 to facilitate securing the patch assembly 230 to an inserter device, as described below. The locking elements 242 may be located on the bottom plate 240 of the patch mount 234. In the illustrated embodiment, the locking elements 242 are in the form of raised headed studs, with a stem 242a and a head 242b, projecting upward from the bottom plate 240 of the patch mount 234. Other variations for the locking elements 242 are possible.

In the illustrated embodiment, the bottom plate 240 of the patch mount 234 also has a raised hub 244 for receiving and retaining a cannula assembly 260. The raised hub 244 is in the form of a cylindrical wall, although other shapes are possible. A locking element 245 in the form of a projection facing inwardly from the inner surface of the wall or hub 244 facilitates retention of the cannula assembly 260.

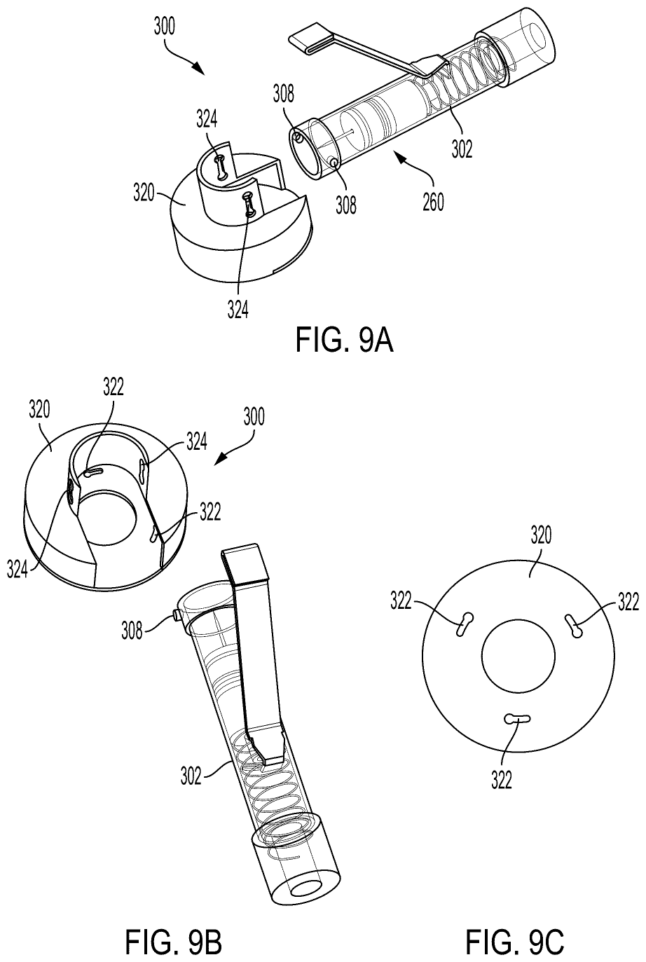

FIGS. 9A and 9B show an example embodiment of certain components of an inserter system 210 (see, e.g., FIG. 12A). The inserter system 210, as supplied, includes an inserter device 300, the patch assembly 230 secured to the bottom of the inserter device 300, and a cannula assembly 260 located within the inserter device 300. FIG. 9C shows a bottom view of a retainer component 320 of the inserter device 300.

As shown in FIGS. 9A and 9B, the inserter device 300 includes a housing 302 which in the illustrated embodiment is in the form of a tube. The inserter device 300 includes a retainer 320 attached to the distal end of the housing 302 in a manner such that the housing 302 can be pivoted with respect to the retainer 320. In the illustrated embodiment, the housing 302 has laterally-extending connecting elements such as tabs or lugs 308 that connect with corresponding connecting elements such as recesses, slots, or openings 324 in a wall of the retainer 320.

As shown in FIG. 9C, the retainer 320 has locking elements 322, which may be located in or on a bottom surface of the retainer 320. The locking elements 322 cooperate with the locking elements 242 of the patch assembly 230 to allow the inserter device 300 to be connected and disconnected from the patch assembly 230. In the illustrated example, the locking elements 322 are keyholes or shaped openings, including a slot with an enlarged area on one end. The locking elements or keys 242 of the patch assembly 230 are shaped such that the heads 242b of the studs 242 can fit through the enlarged area of the keyholes 322 but not through the narrow part of the slots of the keyholes 322. The inserter device 300 is connected to the patch assembly 230 by placing the enlarged openings of the locking elements 322 over the studs 242 and then rotating the inserter device 300 so that the heads 242b of the studs 242 are locked in place behind the narrow parts of the slots 322. The inserter device 300 is disconnected from the patch assembly 230 by the reverse operation. Many other locking element variations are possible for allowing the inserter device 300 to be connected and disconnected from the patch assembly 230. In the illustrated embodiment, the retainer 320 has three locking elements 322 equally spaced at 120 degree increments; however, any suitable number and positioning of locking elements 322 may be used.

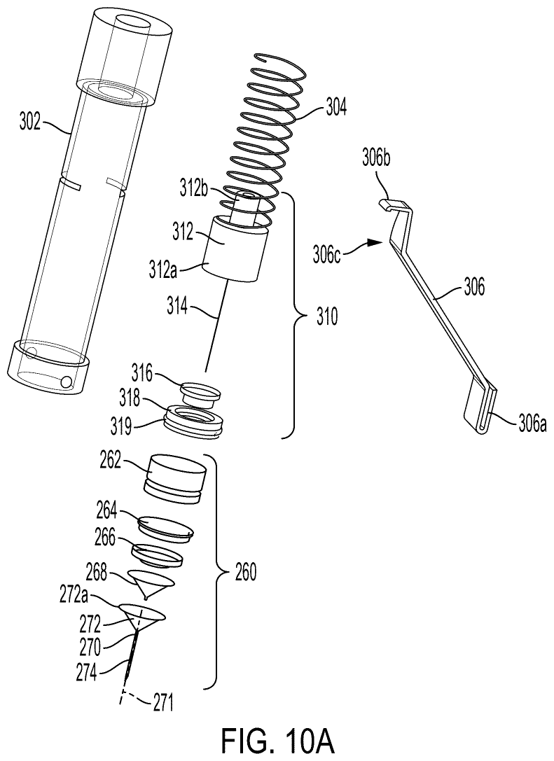

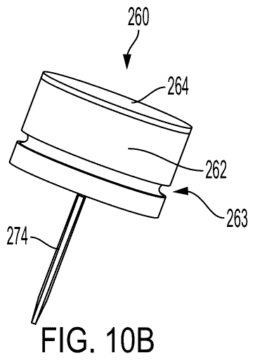

FIG. 10A shows an exploded view of certain components of the example inserter system 210. The inserter device 300 includes the housing 302, the retainer 320 (not shown in FIG. 10A), a spring 304, a trigger 306, and a piston-needle assembly 310. The piston-needle assembly 310 includes a piston 312, a hard needle 314, a platform 316, a stop housing 318, and a retention ring 319. The hard needle 314 may be, for example, stainless steel or other suitable metal. The spring 304 is located inside the tubular housing 302 with its proximal end arranged to be biased against the housing 302 and with its distal end arranged to be biased against the piston 312. The piston 312 may be a cylindrical element adapted to be driven distally by the spring 304. The piston 312 may have a main body 312a at a diameter at or close to the internal diameter of the tubular housing 302 and a stem 312b at a narrower diameter adapted to fit within the coils of the spring 304. This configuration of the piston 312 can help keep the piston 312 aligned within the housing 302. Other configurations are possible.

The hard needle 314 is attached to the piston 312 or, alternatively, to a component driven by the piston 312. When the inserter device 300 is assembled, the platform 316, stop housing 318, and retention ring 319 are positioned inside the tubular housing 302 distal to the piston. The hard needle 314 passes through the platform 316, stop housing 318, and retention ring 319. The stop housing 318 may have a groove or recess for accommodating the retention ring 319, to keep it in position. The retention ring 319 may be an elastomeric ring or gasket adapted to provide some sliding resistance against the inner wall of the tubular housing 302.

When the inserter device 300 is assembled, the spring 304 is compressed and the remaining components inside the tubular housing 302 are in a loaded position. The trigger 306 holds the spring 304 by directly contacting the spring 304 or another component that can keep the spring 304 compressed. The trigger 306 is adapted to allow a user to actuate the inserter device 300 by releasing the spring 304 to drive the piston-needle assembly 310 and cannula assembly 260 distally. The trigger 306 includes a first end 306a, a second end 306b, and a fulcrum 306c. In use, the user (e.g., patient) presses the first end 306a of the trigger 306, which causes the trigger to pivot about fulcrum 306c, thereby moving the second end of the trigger 306b in order to release the spring 304 from its compressed condition.

The inserter system 210 of FIG. 9A also includes a cannula assembly 260, shown in exploded view in FIG. 10A and assembled in FIG. 10B. The cannula assembly 260 includes a cannula 270 having a distal end adapted to be inserted into a patient for drug delivery. In the illustrated embodiment, the cannula 270 has a distal tubular portion 274 and a proximal conical portion 272. In other embodiments the cannula 270 may be only tubular or another suitable configuration. The cannula 270 has a cannula axis 271 that is the axis of the tubular portion 274. Relative to the hard needle 314, the cannula 270 (or at least the tubular portion to be inserted subcutaneously into the patient) may be a relatively soft material, such as a polymer.

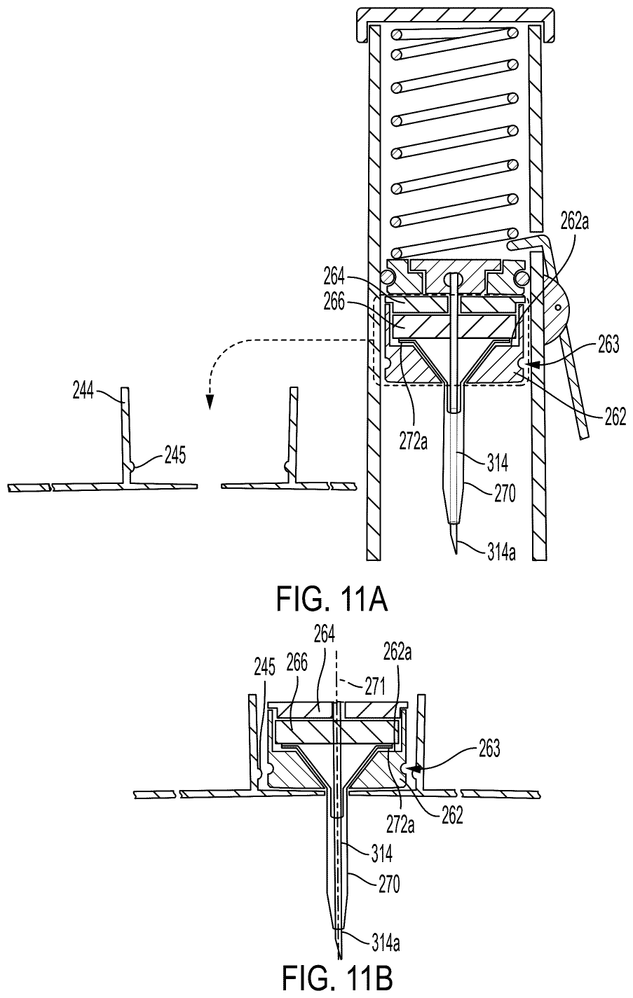

The cannula assembly 260 further includes a housing which may be one or more parts. In the illustrated example, the cannula assembly housing includes a lower housing 262 and a cap or upper housing 264. When the cannula assembly 260 is assembled, a septum 266, a funnel 268, and the proximal end of the cannula 270 are held within the housing. For example, the inside surface of the lower housing 262 may have a flange or ledge 262a (shown in FIGS. 11A and 11B) that holds a lip 272a of the proximal end of the cannula 270 and prevents the cannula from distal movement relative to the housing. The funnel 268 is positioned above the cannula 270, and the septum 266 is positioned above the funnel 268. The cap 264 closes off the top of the housing and prevents the septum 266, funnel 268, and cannula 270 from proximal movement relative to the housing. In this way, the housing components 262, 264 hold the septum 266, funnel 268, and cannula 270 in place. The cap 264 may be firmly secured (e.g., by ultrasonic or heat welding, adhesive, etc.) to the lower housing 262.

The housing of the cannula assembly 260 may further include a locking element 263 for locking the cannula assembly 260 to the patch assembly 230. In the illustrated example, the locking element 263 is a recess adapted to engage the projection 245 on the inner surface of the wall or hub 244 of the patch mount 234, in order to lock the cannula assembly 260 to the patch assembly 230.

When the inserter device 300 is assembled and in the loaded condition (e.g., FIGS. 11A, 12A), the spring 304 is compressed and held by the trigger 306, while the piston-needle assembly 310 and the cannula assembly 260 are in a first, proximal position within the tubular housing 302. The hard needle 314 passes through the platform 316 (the platform 316 may have a central hole, not shown) and stop housing 318 (which may have a hollow center). The hard needle 314 also passes through the cap 264 (the cap may have a central hole, not shown), the septum 266 (which is pierceable by the hard needle 314), the funnel 268, and cannula 270. The sharp distal tip 314a of the hard needle 314 extends out of the distal end of the cannula 270.

FIG. 11A shows a cross-sectional view of certain components of the inserter system 210, prior to attaching the cannula assembly 260 to the patch assembly 230. FIG. 11B shows a cross-sectional view of the cannula assembly 260 attached to the patch assembly 230. As described above, the hard needle 314 may be attached to the piston 312 or, alternatively, to a component driven by the piston 312, such as the platform 316 (in which case the hard needle 314 passes through the stop housing 318 and retention ring 319). When the cannula assembly 260 is attached to the patch assembly 230 (FIG. 11B), the locking element 263 of the lower housing 262 of the cannula assembly 260 is engaged by the projection 245 on the inner surface of the wall or hub 244 of the patch mount 234, thereby locking the cannula assembly 260 to the patch assembly 230.

The following is a description of an example embodiment of a method of using a drug delivery system in accordance with the disclosure. The example drug delivery system 100 is supplied to a user (e.g., a patient) in one or more parts. For example, a pump assembly 120 may be provided with the cartridge assembly 160 loaded into the controller module 130 or with the cartridge assembly 160 separate from the controller module 130. The inserter system 210 may be supplied as a separate component from the pump assembly 120 (or its components). The inserter system 210 may be supplied with the cannula assembly 260 loaded in the inserter device 300 and with the actuating mechanism of the inserter device 300 (spring 304, trigger 306) in the loaded condition. The inserter system 210 may be supplied with the inserter device 300 connected to the patch assembly 230, e.g., through the locking elements 242, 322. Alternatively, the inserter system 210 may be supplied with the inserter device 300 separate from the patch assembly 230, and the user may connect the inserter device 300 to the patch assembly 230, e.g., by the locking elements 242, 322.

FIG. 12A shows a partially-exploded view of an example inserter system 210, with an inserter device 300 in a first position, with the tubular housing 302 pivoted with respect to the retainer 320, so that the tubular housing 302 is generally horizontal or parallel with the patch 232. The inserter device 300 is in its loaded condition, with the cannula assembly 260 loaded in the inserter device 300 and with the spring 304 held compressed by the trigger 306. Either as supplied to the user, or as handled by the user, the inserter device 300 is connected to the patch assembly 230, e.g., by the locking elements 242, 322. In an adhering step, the patient peels the paper backing off of the underside 232b of the patch 232 and adheres the patch 232 to the patient's skin at the desired location (e.g., abdomen, back, arm, shoulder, etc.).

Next, the patient moves the inserter device 300 to its second position, as shown in FIG. 12B, by pivoting the tubular housing 302 with respect to the retainer 320 until the tubular housing 302 is generally vertical or perpendicular to the patch 232. The adhering step, in which the patient peels the paper backing off of the underside 232b of the patch 232 and adheres the patch 232 to the patient's skin at the desired location, may be done before or after the tubular housing 302 is positioned to be generally vertical or perpendicular to the patch 232.

With the tubular housing 302 positioned generally vertical or perpendicular to the patch 232, the patient presses the first end 306a of the trigger 306, which causes the trigger 306 to pivot about the fulcrum 306c and moves the second end 306b of the trigger to release the spring 304 from its compressed condition. When released, the spring 304 drives the piston-needle assembly 310 and the cannula assembly 260 distally until the cannula assembly 260 is locked to the patch assembly 230, e.g., by the engagement of locking element 263 with locking element 245. Thus, by actuation of the trigger 306, the patient launches the cannula assembly 260 for deployment and locking to the patch assembly 230.

When the spring 304 drives the piston-needle assembly 310 and the cannula assembly 260 distally, the force causes the sharp distal end 314a of the hard needle 314 to pierce the patient's skin. The spring 304 drives the hard needle 314 and the tubular distal end of the cannula 270 into the skin to a sufficient depth at which a drug can be effectively delivered to the patient from the distal end of the cannula 270.

Forward motion of the piston-needle assembly 310 and the cannula assembly 260 is stopped by the engagement of the cannula assembly housing 262 with the patch mount 234. Additionally or alternatively, forward motion of the piston-needle assembly 310 and the cannula assembly 260 may be stopped by the friction resistance of the retention ring 319 with the inner surface of the tube 302, and/or by a positive stop feature such as a stop ledge on the inside of the tube 302.

After the inserter device 300 has been actuated, with the cannula 270 inserted in the patient's skin and the cannula assembly 260 engaged with the patch assembly 230, the patient can safely remove the inserter device 300. In the illustrated embodiment, the patient removes the inserter device 300 from the patch assembly 230 by rotating it relative to the patch assembly 230 so that the locking elements 322 disengage from the locking elements 242. Then, the inserter device 300 can be removed, leaving the patch-cannula assembly 220 (i.e., the patch assembly 230 with the cannula assembly 260 engaged with it) adhered to the patient (see reference numeral 220 in FIG. 12C). The hard needle 314 is withdrawn from the cannula 270 and safely shrouded within the inserter device 300. The cannula 270 remains inserted in the patient's skin. The inserter device 300 can be recycled, discarded, or returned for sterilization and reuse.

FIG. 12C shows the example drug delivery system 100, after deployment of the cannula assembly 260 and separation of the inserter device 300 from the patch-cannula assembly 220. FIG. 12D shows the underside of the patch-cannula assembly 220 after deployment of the cannula assembly 260. As shown in FIGS. 12C and 12D, the cannula assembly 260 is engaged with the patch assembly 230, while the underside of the patch 232 is adhered to the patient's skin and the cannula 270 projects beyond the patch 232 and is inserted into the patient's skin.

With the patch-cannula assembly 220 adhered to the patient and the cannula 270 in position for drug delivery, the patient can then mount the pump assembly 120 on the patch-cannula assembly 220 to form the completed wearable drug delivery device 110. The cartridge assembly 160 can be supplied to the patient pre-filled with a drug fluid, or the patient can fill the cartridge assembly 160 with the drug fluid. To fill the cartridge assembly 160, the patient inverts the cartridge assembly 160 so that the fill port 168 is on top. The patient takes a syringe (not shown) and fills the syringe to a predetermined, indicated, or desired level with drug fluid, typically from a vial (not shown). The patient takes the syringe filled with drug fluid and inserts the needle of the syringe through the fill septum 169 and fill port 168 and into the chamber of the reservoir 164. The patient then dispenses the drug fluid from the syringe into the reservoir 164. During filling, the fluid outlet port 167 can serve as a vent allowing air to escape. Alternatively, one or more vents may be provided in the wall of the reservoir 164. Once the desired drug fluid is dispensed into the reservoir 164, the patient removes the needle of the syringe from the fill septum 169 and fill port 168. The patient then attaches the filled cartridge assembly 160 to the controller module 130 to complete the pump assembly 120. In certain embodiments, the connection of the cartridge assembly 130, which houses the battery 198, to the controller module 130, which houses the electronics, connects the battery 198 to the electronics and “wakes up” or turns on the pump assembly 120.

The patient attaches the pump assembly 120 to the patch-cannula assembly 220 by placing the pump assembly 120 on the patch-cannula assembly 220 so that the ridge 236 of the patch mount 234 receives the ridge 136 of the controller module 130 and so that the ridge 238 of the patch mount 234 receives the ridge 138 of the controller module 130. The locking elements 137 of the controller module 130 engage with the locking elements 237 of the patch mount 234 to secure the removable pump assembly 120 to the patch-cannula assembly 220. FIG. 12E shows the assembled wearable drug delivery device 110, with the upper housing and certain components of the controller module removed to show internal features of the pump assembly 120.

When the removable pump assembly 120 is connected to the patch-cannula assembly 220, the tube 166 from the reservoir 164 is aligned to dispense drug fluid from the reservoir 164 into the funnel 268 and cannula 270. The tube 166 may pierce the septum 266 of the cannula assembly 260 so that the fluid outlet port 167 dispenses the drug fluid into the funnel 268 and cannula 270 and consequently into the patient.

As mentioned above, the electronics in the removable pump assembly 120 may receive input data from an external source, such as from a smartphone, tablet, computer, or remote source. The input data may include signals for controlling the functioning of the wearable drug delivery device and may include information such as the timing, frequency, and amounts of doses to be administered.

The external source (smart phone, tablet, computer, etc.) may send this information to the removable pump assembly 120 based on input to an application or computer program from a physician and/or patient. Such input may be based on various factors such as a medical condition, age, weight, lifestyle, anticipated activity, and/or any other relevant factor(s). The input may include a dosing regimen. The input may also include a feature allowing the user to alter the regimen (e.g., deliver an additional dose) if advisable (e.g., based upon anticipated activity, glucose level, etc.). The external source may also receive real-time or near real-time information from another source, such as from a glucose monitor, and may incorporate this information in determining the signals sent to the removable pump assembly 120. As mentioned above, a glucose monitor may be incorporated into the removable pump assembly 120 or may be another device; in either event the glucose monitor may communicate with the external source that sends signals for controlling the functioning of the wearable drug delivery device.

The external source (smart phone, tablet, computer, etc.) may also receive input from the wearable drug delivery device. For example, it may receive information from the pressure sensor(s), the encoder(s), and/or one or more other sensors, and/or from the battery. The external source may use this input in a similar manner as described above with respect to the electronics of the wearable drug delivery device. For example, it may activate a signal upon detection of an occlusion, depletion of the reservoir, low battery, etc.

When attached to the patient, the wearable drug delivery device 110 serves to deliver automatically and periodically the desired drug amounts to the patient. During operation, the various inputs described above are received, the dosing is controlled, and the various signals described above may be activated depending on the inputs and programming.

Once the drug fluid in the cartridge assembly 160 is depleted (fully dispensed or dispensed leaving drug fluid below a certain level), the patient can remove the cartridge assembly 160. The patient removes the pump assembly 120 from the patch-cannula assembly 220 by pulling the pump assembly 120 off of the patch-cannula assembly 220 so that the locking elements 137 of the controller module 130 disengage from the locking elements 237 of the patch mount 234. The patient then disengages the cartridge assembly 160 from the controller module 130. Then, the patient may discard the cartridge assembly 160. In some embodiments, if suitable for the particular drug and application, the patient may reuse the cartridge assembly by refilling the reservoir 164 with drug fluid (using a separate drug-filled vial and syringe) in the same manner as described above. Otherwise, the patient may get a new cartridge assembly 160 and fill the reservoir 164 with drug fluid as described above. The patient then connects the filled cartridge assembly 160 with the controller module 130 and attaches the pump assembly 120 to the patch-cannula assembly 220 in the same manner as described above.

In certain embodiments, the controller module 130 may be reusable, and the cartridge assembly 160 may be disposable after a single use or disposable after a certain number of refills or after a certain period of time. In one example, the controller module may be reusable for 24 to 36 months, or longer. In one example, a single cartridge assembly may hold enough drug fluid to last for 3-7 days, or longer. Other device life-spans are possible.

Wearable drug delivery devices as disclosed herein may be used for delivery of any suitable therapeutic drug for any suitable condition. In one example, wearable drug delivery devices as disclosed herein may be used for the delivery of insulin for the treatment of diabetes. Other drugs and conditions are possible, such as for treating or managing cholesterol, heart disease, high blood pressure, hormone levels, and numerous other possibilities.

Wearable drug delivery devices as disclosed herein may be adhered at any suitable location on a patient's skin. Examples include, but are not limited to, the patient's abdomen, shoulder, arm, and back.

In certain embodiments, and as a significant departure from certain prior patch pumps in commercial use, the reservoir of the cartridge assembly can have a broad, flattened profile. This allows the reservoir to have a good fill volume as well as a low profile, allowing the overall pump device to have a low profile on the patient's body.

Thus, in certain embodiments, the reservoir can have a larger cross-sectional area relative to certain prior patch pumps (the cross-sectional area is along a cross-section taken perpendicular to the axis 165 of the reservoir). Similarly, the reservoir can have a relatively large diameter, width, or cross-sectional area (measured perpendicular to the axis 165 of the reservoir) as compared to its height (measured in the direction of the axis 165 of the reservoir).

In the illustrated example, the threaded actuators 194, 196 are aligned along a central longitudinal axis 191, perpendicular to the general plane of the piston disc 180. The actuator axis 191 is aligned parallel with the reservoir axis 165. In the illustrated example, the actuator axis 191 is colinear with the reservoir axis 165; in other embodiments, the actuator axis 191 may be offset from the reservoir axis 165. The reservoir 164 has a cross-sectional area perpendicular to its axis 165. While the reservoir may have any suitable size, in some embodiments the diameter, width, or cross-sectional area of the reservoir (measured perpendicular to the reservoir axis 165) can be relatively large compared to the height of the reservoir (measured along reservoir axis 165). For example, in some examples, the inside width or diameter of the reservoir chamber may be in the range of 0.70 inches to 1.20 inches, and the depth of the reservoir chamber may be in the range of 0.20 inches to 0.35 inches, e.g., 0.22 to 0.25 inches. Thus, in certain embodiments, the ratio of the diameter or width of the reservoir chamber to the depth of the reservoir chamber may be 2:1 or greater, 3:1 or greater, 4:1 or greater, 5:1 or greater, or 6:1 or greater. This results in a broad, flattened profile, with good fill volume capacity. While the fill volume capacity of the reservoir may be any suitable fill volume, in some examples the reservoir volume is in the range of 1 ml to 3 ml. Other volumes are possible.

In conjunction with the reservoir having a relatively large diameter, width, or cross-sectional area as compared to its height, in certain embodiments (like the reservoir 164), the reservoir can be oriented in the device so that the reservoir axis 165 is parallel to the cannula axis 271. In certain embodiments, the reservoir axis 165 may be colinear with the cannula axis 271. Thus, when the pump is mounted on a patient, the reservoir axis 165 is aligned vertical to (or perpendicular to) the patch adhered to the patient's skin. This is opposed to certain prior devices in which the axis of a reservoir is aligned perpendicular to the cannula axis and horizontal to (or parallel to) the patient's skin. With the reservoir axis 165 aligned parallel to the cannula axis 271, or vertical to (or perpendicular to) the patch on patient's skin, the actuator axis 191 may also be aligned parallel to the cannula axis 271, or vertical to (or perpendicular to) the patch on patient's skin.

With the orientation and relative dimensions as described above, the reservoir can have a broad, flattened profile on the patient's body. This can be analogized to a hockey puck shape, or pancake shape, or disc shape, or flat shape, or flattened shape, lying flat on the patient's skin.

A reservoir with a large cross-sectional area and with a broad, flattened profile can present challenges with respect to maintaining accuracy, precision, and consistency of drug delivery. The inventions disclosed herein include, in some embodiments, the use of certain novel aspects to solve these challenges. These novel aspects include aspects relating to materials and manufacturing that depart significantly from certain prior patch pumps in commercial use.

Thus, in certain embodiments of the invention, the reservoir of the cartridge assembly may be manufactured from thin sheet metal, using precision manufacturing as disclosed herein. Previously, patch pumps have employed injection-molded plastic reservoirs, and in some embodiments of the disclosure the reservoir may be made of injection-molded plastic. However, in some embodiments of the present disclosure, the precision manufacturing of sheet metal can be used, and such material and manufacturing can be particularly advantageous for drug reservoirs sized and shaped as described herein. If desired, the sheet metal may be coated with a coating compatible with the intended drug (e.g., insulin).

In accordance with some embodiments of this disclosure, the reservoir can be manufactured using a metal deep-draw process. The process may include the use of progressive dies that successively form the metal in a sequence of steps from the sheet to the final shape. Such a deep-draw process allows precision formation of the reservoir features, such as ribs on the inside of the reservoir wall, to tight tolerances. The ribs serve as guide rails to help prevent the piston disc from tilting during the fluid dispensing process. Precision manufacturing of the ribs facilitates precision drug delivery. The deep-draw process also facilitates formation of the microminiature tube at the bottom of the reservoir chamber. Other metal manufacturing techniques may be used, such as metal forming, stamping, chemical etching, EDM processes, laser cutting, laser welding, high temperature and high pressure forging, metallized molding, 3D printing, or other metal manufacturing processes capable of creating intricate features on a small metal part with high precision, accuracy, and tight tolerances, with consistency. The metal manufacturing technology permits the use of certain automated manufacturing processes, including, for example, during raw metal material prepping, coating, stamping, deep-drawing, and laser welding. Such automated manufacturing processes can reduce manufacturing costs.

The use of thin sheet metal and manufacturing as disclosed herein can produce a reservoir as disclosed herein, with a low profile and good fill volume, capable of accurate, precise, and consistent drug delivery. The materials and manufacturing disclosed herein are capable of manufacturing tolerances of 0.0005 inches or better or in some cases 0.0002 inches or better. The tolerances achievable with the materials and manufacturing disclosed herein generally have not previously been achievable with the types of plastic injection-molded reservoirs in certain prior wearable patch pumps.

Other novel aspects that may be employed to facilitate the use of a reservoir with a large cross-sectional area and a broad, flattened profile relate to the actuator assembly. The large cross-sectional area of the reservoir can necessitate a piston disc with a large diameter or width. The large piston disc can necessitate a large contact engagement between the seal at the perimeter of the piston disc and the inner wall of the reservoir. This can create increased friction and resistance to movement. The large piston disc and associated features can make the piston disc susceptible to tilting and inaccurate, imprecise, or inconsistent drug delivery.

In certain embodiments, a large diameter threaded actuator is used, which can help prevent tilting of the piston disc. For example, in certain embodiments, the ratio of the diameter of the threaded actuator attached to the piston disc to the width or diameter of the reservoir chamber may be from 1:4 to 1:2 or 2:3. That is, the diameter of the threaded actuator attached to the piston disc may be from one-quarter to one-half or two-thirds of the diameter or width of the reservoir chamber. In alternate embodiments, the diameter of the threaded actuator attached to the piston disc may be one-quarter or more of the diameter or width of the reservoir chamber. In alternate embodiments, the diameter of the threaded actuator attached to the piston disc may be one-half or more of the diameter or width of the reservoir chamber. In alternate embodiments, the diameter of the threaded actuator attached to the piston disc may be two-thirds or more of the diameter or width of the reservoir chamber. An example is a piston disc diameter of 1.0 inches (corresponding to a diameter or width of the reservoir chamber of approximately 1.0 inches) and a threaded actuator diameter of 0.25 inches to 0.50 inches or 0.60 inches or 0.65 inches. Other dimensions and ratios are possible. The use of a large threaded actuator diameter facilitates high axial directional accuracy for use with the large diameter piston disc. The use of a large threaded actuator diameter helps prevent the piston disc from tilting during the fluid dispensing process.

The use of a large diameter threaded actuator can also be advantageous in that the lower threaded actuator can be directly mounted to the piston disc without the need for any additional device or component to support the threaded actuator in a true-center position relative to the center of the piston disc. Similarly, the upper threaded actuator can be directly mounted to the toothed wheel. The threaded actuator(s) can be directly welded (e.g., tack or spot welded) by any suitable technique (e.g., resistance, laser, ultrasonic, and/or heat welding) or otherwise bonded or adhered (e.g., with epoxy) to the piston disc and/or toothed wheel. In some alternative embodiments, one or more additional components may be used to attach or support the threaded actuator(s) on or relative to the piston disc and/or toothed wheel.

While a large diameter threaded actuator can have the stated advantages, it also can create increased thread contact area and, resultingly, increased friction for the actuator assembly to overcome. This resistance and the resistance from the seal around the large piston disc can create a high force for an actuator drive to overcome. Thus, in accordance with certain embodiments herein, the electromechanical drive system 140 employs a worm gear 144. The worm gear system provides a higher torque in a compact area. An electromechanical drive system 140 with a worm gear 144 can help prevent the threaded actuator assembly from binding to the gear motor during rotation. In accordance with certain embodiments herein, a large volume, low profile reservoir may be combined with a small motor with high torque. The features described herein facilitate a low profile device with precision drug delivery.

Also, in certain embodiments, the threaded actuator is provided with precision fine threads, which facilitate minute, precise movements of the piston disc for precise drug delivery. In some embodiments, the first threaded actuator 194 and the second threaded actuator 196 may have precision fine threads at a density of 80 threads per inch (tpi) or more, 100 tpi or more, 300 tpi or more, 450 tpi or more, or 500 tpi or more. The first threaded actuator 194 and the second threaded actuator 196 may be made of any suitable materials, such as metal (e.g., carbon, stainless steel, cobalt, brass, titanium), plastic, etc.

Another feature that may be employed with certain embodiments is that the pump housing (housing of the controller module) may be translucent or transparent, such as a clear plastic, in whole or in part. Artwork can be laminated, printed, engraved, etched, laser engraved, and/or molded onto an inner surface of the housing. The controller module housing can also be left alone as a transparent/clear housing, so that the inner working of the device, and/or internal artwork, can be seen. This can enhance the aesthetics of the device, avoid external decoration or artwork which could be scratched or otherwise damaged, and consequently increase user compliance with wearing the device. In alternative embodiments, the pump housing is decorated with artwork, is of a solid color, is multi-colored, or has any other aesthetically suitable appearance.

In an example embodiment, the outer diameter of the controller module may be about 2.0 inches or less, e.g., 1.7 to 1.8 inches, and the height of the controller module may be about 0.8 inches or less, e.g., 0.5 to 0.7 inches. The wall thickness of the controller housing may be about 0.040 inches to about 0.060 inches. The inserter device housing may have a diameter of about 0.25 inches. Many other dimensions are possible.

The components of a drug delivery system as described above may be manufactured from any suitable material, including polymers and metals, such as stainless steel and titanium. Any suitable manufacturing process may be used. For example, the controller module housing, the cartridge assembly housing, the reservoir, and/or the patch mount may be injection molding or thermal formed or vacuum formed or 3D printed from a suitable plastic material. Any septum may be an elastomer such as silicone or rubber. The funnel of the cannula assembly may be metal. The hard needle may be stainless steel or another metal. The cannula or soft needle may be a polymer. Many other variations are possible.

Embodiments of a system, device, assembly, or method within the scope of the disclosure may have one or more advantages, such as, but not limited to: small size, small footprint, low profile, flat profile, light weight, large reservoir internal volume, aesthetic appeal, wearability, absence of external tubes outside of the device housing, convenience of use, ease to use, accuracy in drug delivery, precision in drug delivery, consistency in drug delivery, low cost, manufacturability in high volumes, and economic manufacturability. Embodiments of a wearable drug delivery device (wearable infusion cannula patch pump) may be small, inconspicuous, discreet, and unobtrusive, resulting in advantages of comfort, aesthetics, usage, and compliance. Embodiments may have a low profile while maintaining a large volume reservoir and accuracy, precision, and consistency in drug delivery.

Persons of ordinary skill in the art will appreciate that the embodiments encompassed by the disclosure and claims are not limited to the example embodiments illustrated and described above. Numerous other variations, modifications, changes, and substitutions are possible and contemplated within the scope of the disclosure and claims, as would be understood by persons of ordinary skill in the art.

1. A wearable drug delivery device comprising:
a removable pump assembly comprising a cartridge assembly and a controller module;
a patch assembly; and
a cannula assembly comprising a cannula having a cannula axis;
wherein the cartridge assembly comprises a reservoir having a reservoir chamber and a reservoir axis;
wherein the reservoir is oriented in the cartridge assembly such that when the pump assembly is attached to the patch assembly, the reservoir axis is aligned parallel to the cannula axis.

2. The wearable drug delivery device according to claim 1, wherein the ratio of the diameter or width of the reservoir chamber to the depth of the reservoir chamber is 2:1 or greater.

3. The wearable drug delivery device according to claim 1, wherein the controller module has a housing that is translucent or transparent.

4. The wearable drug delivery device according to claim 1, further comprising an encoder.

5. The wearable drug delivery device according to claim 1, further comprising a pressure sensor.

6. A wearable pump assembly comprising:
a cartridge assembly; and
a controller module;
wherein the cartridge assembly comprises a reservoir;
wherein the reservoir has been manufactured from sheet metal.

7. The wearable pump assembly according to claim 6, wherein the reservoir has been manufactured using a deep draw process.

8. The wearable pump assembly according to claim 6, wherein the reservoir comprises one or more internal ribs.

9. The wearable pump assembly according to claim 8, further comprising a piston disc, wherein the piston disc has one or more notches for accommodating the one or more ribs of the reservoir.

10. The wearable pump assembly according to claim 9, further comprising a seal gasket around the perimeter of the piston disc, wherein the seal gasket has one or more notches for accommodating the one or more ribs of the reservoir.

11. A wearable pump assembly comprising:
a cartridge assembly; and
a controller module;
wherein the cartridge assembly comprises a reservoir having a reservoir chamber, a piston disc, and an actuator assembly having a threaded actuator attached to the piston disc;
wherein the diameter of the threaded actuator attached to the piston disc is one-quarter or more of a diameter or width of the reservoir chamber.

12. The wearable pump assembly according to claim 11, further comprising an actuator retainer for maintaining alignment of the actuator assembly.

13. The wearable pump assembly according to claim 11, further comprising a drive system with a worm gear for driving the actuator assembly.

14. The wearable pump assembly according to claim 11, wherein the threaded actuator has a thread density of 80 threads per inch or higher.

15. The wearable pump assembly according to claim 11, wherein the diameter of the threaded actuator attached to the piston disc is one-half or more of the diameter or width of the reservoir chamber.

16. An inserter system for a drug delivery system, the inserter system comprising:
an inserter device; and
a cannula assembly loaded in the inserter device.

17. The inserter system according to claim 16, wherein the inserter device comprises a retainer adapted to be selectively attached to a patch assembly.

18. The inserter system according to claim 17, wherein the inserter device is adapted to launch the cannula assembly so that the cannula assembly connects to the patch assembly.

19. (canceled)

20. (canceled)
